(19) United States
(12) Patent Application Publication
(10) Pub. No.: US 2010/0324517 A1
(43) Pub. Date: Dec. 23, 2010

(54) Absorbent Article Having Size Fit Indicating Means

(75) Inventors: Karoline Lenhult, Alvangen (SE); Cilla Persson, Goteborg (SE)

Correspondence Address: BUCHANAN, INGERSOLL & ROONEY PC, POST OFFICE BOX 1404, ALEXANDRIA, VA, 22313-1404, US

(73) Assignee: SCA Hygiene Products AB, Goteborg (SE)

(21) Appl. No.: 12/667,078

(22) PCT Filed: Jun. 29, 2007
(86) PCT No.: PCT/SE07/00637

§ 371 Date: Dec. 29, 2009

(51) Int. Cl. A61F 13/496; A61F 13/49

(52) U.S. Cl. 604/385.01; 604/385.03

(57) ABSTRACT

Absorbent article in the form of a diaper or incontinence guard includes a first and second body panels and a target area arranged on the first body panel intended to receive fasteners located at opposite longitudinal side edges of the second body panel. The target area is located in a transverse central part of the first body panel and at least one size fit indicating means is located between the target area and a longitudinal side edge of the first body panel. The size fit indicating means is adapted to indicate that the article is too small for the wearer. The article may form a part of an assortment of different sizes of articles, each size having a selected indication in the form of colour, character, graphic, text or combinations thereof.

### TECHNICAL FIELD

The present invention refers to an absorbent article in the form of a diaper or incontinence guard of the type that comprises first and second body panels and a crotch portion therebetween, said first body panel comprises a target area intended to receive fasteners located at the opposite longitudinal side edges of the second body panel. When the fasteners are fastened to the target area, the article will assume a pant-like shape. The article further comprises at least one size fit indicating means arranged on the first body panel.

### BACKGROUND OF THE INVENTION

Absorbent articles of the above mentioned kind comprises fasteners, usually in the form of adhesive tape or hook material, attached to opposite longitudinal sides of one of the body panels, usually the back body panel, and intended to be fastened to a target area on the opposite body panel. This target area may comprise a strip of material adapted to receive the fastener or may, in case the outside material of the body panel is adapted to receive the fasteners, simply be a portion of the body panel, usually the front body panel, said portion is intended to receive the fasteners for a correct fit of the article. The target area may be indicated by colour, characters, graphic, text or combinations thereof in order for the caregiver to easily attach the fasteners in a correct position to provide a proper fit of the article, since in order to obtain a good security against leakage a reasonably tight fit on the wearer is necessary. If the size of the article is too small for the wearer this may also result in leakage problem and ill-fitting of the article.

US 2007/0049896 discloses an absorbent article having a size fit indicating means for indicating when the article is too large for a wearer. Two target strips for fasteners are provided on the front body panel and a visual sizing indicator is positioned between the two target strips, so that when the fasteners overlap the sizing indicator this is an indication of that the article is too large.

U.S. Pat. No. 5,133,707 discloses a diaper having an adhesive fastening tape system including a transparent fastening tape and a target area on the front body panel having indica means in the form of characters, numbers, letters or the like.

U.S. Pat. No. 5,897,546 discloses a diaper having fastening system comprising tape fasteners and a target area in the form of a transparent receiving sheet intermittently bonded to the outer sheet of the front body panel by means of an adhesive serving as a positioning indicator for the tape fasteners.

U.S. Pat. No. 6,448,202 discloses a device and method for identifying a size of an absorbent article which is appropriate for a potential wearer based on at least two characteristics, such as weight and height.

WO 03/034966 discloses an absorbent article having fasteners in the form of hook material engageable with the outer nonwoven surface of the front body panel and wherein landing zone graphics are printed in the target area for the fasteners and covered by an outer nonwoven and visible through said outer nonwoven.

Thus there exists prior art teaching the use of indicia for guiding the caregiver to fasten a diaper on a user. However none of this prior art provides clear information of whether the article is of a correct size or is too small for the wearer and needs to be replaced for a larger size in order to provide a satisfactory fit and function. For babies that are growing it is often difficult for the caregiver to determine when a product is actually too small for the wearer and it is time to change to a larger size.

### OBJECT AND MOST IMPORTANT FEATURES OF THE INVENTION

The present invention aims at providing an absorbent article, especially a baby diaper or an incontinence guard, of the kind mentioned above and which solves the problem of indicating to the caregiver whether the article is of a correct size or whether the wearer needs an article of a larger size. This has according to the invention been provided by the fact that the target area for the fasteners is located in a transverse central part of the first body panel and size fit indicating means is located between said target area and a longitudinal side edge of the first body panel and is adapted to indicate that the article is too small for the wearer.

In one aspect of the invention the article is of a selected size forming a part of an assortment of at least two different sizes of articles, wherein each size has a selected indication in the form of colour, character, graphic, text or combinations thereof, said size fit indicating means comprises at least one area having a colour, character, graphic, text or combinations thereof being an indication of a size larger than the size of the article itself. This means that the caregiver in addition to being informed of that the article is too small also is informed of to which size the change should be made.

In a further aspect the target area and the size fit indicating means are arranged so that when, in use, the article is fastened to a wearer by means of said fasteners the respective longitudinal side edge of the second body panel, to which the fastener is attached, will be located laterally inside the size fit indicating means to substantially hide the size fit indicating means when the size of the article is adequate for the wearer, but when the size of the article is too small for the wearer the size fit indicating means will become visible between the target area and said longitudinal side edge of the second body panel.

In one embodiment size fit indicating means are arranged on both lateral sides of the said transverse central part forming the target area.

In a further embodiment the size fit indicating means comprises a transition zone proximate to said target area, wherein said transition zone forms a gradual indication of that the article is too small for the wearer by means of intensity of colour, size, pattern density or expression of characters, graphic, text or combinations thereof.

According to one aspect of the invention the target area and the size fit indicating means comprise a pattern printed on a garment facing surface of the article.

In one embodiment a strip of reception material adapted to receive said fasteners is arranged on said first body panel, said strip extending in lateral direction over said target area and over at least a part of the size fit indicating means, said strip of reception material being transparent so that said printed pattern is visible therethrough.

In an alternative embodiment that the target area is provided on a strip of reception material adapted to receive said fasteners. The size fit indicating means may comprise a pattern printed on said strip of reception material.

In a further embodiment the garment facing surface of the article acts as a reception material adapted to receive said fasteners and wherein the size fit indicating means comprises a pattern printed on the garment facing surface of the article.

In a still further embodiment the target area comprises a pattern and/or colour distinctly different from that of the size fit indicating means.

In a further aspect the absorbent article is a baby diaper.

### BRIEF DESCRIPTION OF THE DRAWINGS

The invention will in the following be closer described with reference to some embodiments shown in the accompanying drawings.

FIG. 1 shows a plan view of a diaper according to one embodiment of the invention seen from its garment facing side.

FIG. 2a illustrates the configuration of the diaper when in use with the fasteners applied in the target area.

FIG. 2b illustrates the configuration of the diaper when in use with the fasteners applied outside the target area thus exposing the size fit indicating means.

FIG. 3 shows a plan view of a diaper as seen from the garment facing side according to a second embodiment.

FIG. 4 shows the first body panel of a further embodiment of the invention

### DESCRIPTION OF PREFERRED EMBODIMENTS

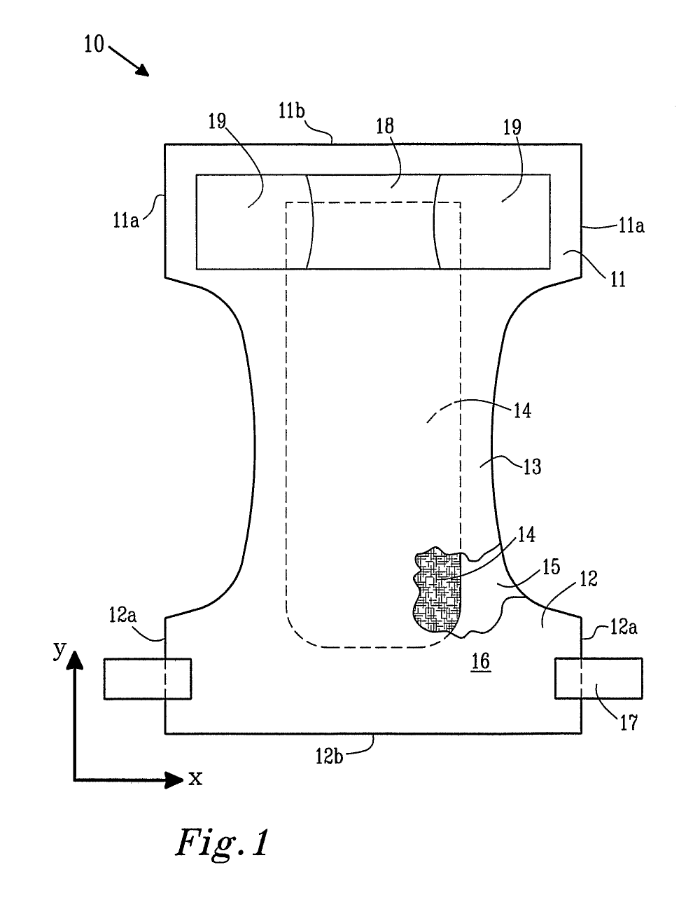

FIG. 1 of the drawings shows an embodiment of an absorbent article in the form of a baby diaper or and incontinence guard 10 comprising a first body panel 11, which in the embodiment shown in the drawings is the part of the article that in use is intended to extend over the stomach and front hip area of the wearer. The article also comprises a second body panel 12, which in the shown embodiment is the part of the pant diaper that in use is intended to extend over the back and the rear hip area of the wearer. It is understood that alternatively the first body panel 11 may be the back body panel and the second body panel 12 may be the front body panel. The article has a longitudinal direction x and a transverse direction y. The first and second body panels have longitudinal and transverse side edges, 11a, 11b and 12a, 12 b respectively.

The crotch portion 13 of a garment 10 is the part of the garment that in use is intended to extend through the wearer's crotch area, between the legs. An absorbent core 14 is disposed in the crotch portion 13 and extends into the front and back body panels 11 and 12. The absorbent core 14 is disposed between an inner coversheet 15 and an outer coversheet 16.

The term “inner coversheet” refers to the liquid permeable material sheet forming the inner cover of the absorbent garment and which in use is placed in direct contact with the skin of the wearer. The inner coversheet can comprise a nonwoven material, e.g. spunbond, meltblown, carded, hydroentangled, wetlaid etc. Suitable nonwoven materials can be composed of natural fibers, such as wood pulp or cotton fibres, man-made fibres, such as polyester, polyethylene, polypropylene, viscose, rayon etc. or from a mixture of natural and man-made fibres. The inner coversheet material may further be composed of tow fibres, which may be bonded to each other in a bonding pattern, as e.g. disclosed in EP-A-1 035 818. Further examples of inner coversheet materials are porous foams, apertured plastic films etc. The materials suited as inner coversheet materials should be soft and non-irritating to the skin and be readily penetrated by body fluid. The inner coversheet may further be different in different parts of the absorbent garment.

The “outer coversheet” refers to the material forming the outer cover of the absorbent garment. The outer coversheet may be the same or different in different parts of the absorbent garment. At least in the area of the absorbent core the outer coversheet comprises a liquid impervious material a thin plastic film, e.g. a polyethylene or polypropylene film, a nonwoven material coated with a liquid impervious material, a hydrophobic nonwoven material, which resists liquid penetration, or a laminate of a plastic film and a nonwoven material. The outer coversheet material may be breathable so as to allow vapour to escape from the absorbent core, while still preventing liquids from passing therethrough. Examples of breathable outer coversheet materials are porous polymeric films, nonwoven laminates of spunbond and meltblown layers and laminates of porous polymeric films and nonwoven materials. Preferably, the outer coversheet comprises a nonwoven material on at least the undergarment-facing surface thereof.

The “absorbent core” is the absorbent structure disposed between the two coversheets of the absorbent garment in at least the crotch region thereof. The absorbent core can be of any conventional kind. Examples of commonly occurring absorbent materials are cellulosic fluff pulp, tissue layers, highly absorbent polymers (so called superabsorbents), absorbent foam materials, absorbent nonwoven materials or the like. It is common to combine cellulosic fluff pulp with superabsorbent polymers in an absorbent core. Superabsorbent polymers are water-swellable, water-insoluble organic or inorganic materials capable of absorbing at least about 20 times their own weight of an aqueous solution containing 0.9 weight percent of sodium chloride. Organic materials suitable for use as superabsorbent materials can include natural materials such as polysaccharides, polypeptides and the like, as well as synthetic materials such as synthetic hydrogel polymers. Such hydrogel polymers include, for example, alkali metal salts of polyacrylic acids, polyacrylamides, polyvinyl alcohol, polyacrylates, polyacrylamides, polyvinyl pyridines, and the like. Other suitable polymers include hydrolyzed acrylonitrile grafted starch, acrylic acid grafted starch, and isobutylene maleic anhydride copolymers and mixtures thereof. The hydrogel polymers are preferably lightly cross-linked to render the material substantially water insoluble. Preferred superabsorbent materials are further surface cross-linked so that the outer surface or shell of the superabsorbent particle, fibre, flake, sphere, etc. possesses a higher crosslink density than the inner portion of the superabsorbent. The superabsorbent materials may be in any form which is suitable for use in absorbent composites including particles, fibres, flakes, spheres, and the like.

A high liquid storage capacity is provided by the use of high amounts of superabsorbent material. For an absorbent core comprising a matrix of hydrophilic fibres, such as cellulosic fibres, and superabsorbent material, the proportion of superabsorbent material is preferably between 10 and 90% by weight, more preferably between 30 and 70% by weight.

It is conventional for absorbent garments to have absorbent cores comprising layers of different properties with respect to liquid receiving capacity, liquid distribution capacity and storage capacity. The thin absorbent bodies, which are common in for example baby diapers and incontinence guards, often comprise a compressed, mixed or layered structure of cellulosic fluff pulp and superabsorbent polymers. The size and absorbent capacity of the absorbent core may be varied to suit different uses, such as infants or adult incontinent persons.

The absorbent core may further include an acquisition distribution layer placed on top of the primary absorbent body, which is adapted to quickly receive and temporarily store discharged liquid before it is absorbed by the primary absorbent core. Such acquisition distribution layers are well known in the art and may be composed of porous fibrous wadding or foam materials.

The article may further contain elastic features, such as leg elastics, waist elastics, raised leakage barriers along the absorbent core etc. which per se are known in the art, but not shown in the drawings.

Fasteners 17 are attached to the each of the longitudinal side edges 12a of the second body panel 12 and are intended to be fastened to the outside of the first body panel 11. The fasteners 12 may comprise an adhesive tape or a mechanical fastener, especially a hook fastener of a hook-and-loop fastening means. A “hook-and-loop fastener” refers to complementary fastening means having a “hook” portion and a “loop” portion and which are refastenable. The term “hook” as used herein refers to any element capable of engaging another element, the so called “loop” portion. The term “hook” is not limited to only “hooks” in its normal sense, but rather encompasses any form of engaging elements, whether unidirectional or bi-directional. The term “loop” is likewise not limited to “loops” in its normal sense, but also encompasses any structure capable of engaging with a “hook” fastener. Examples of “loop” materials are fibrous structures, like nonwoven materials. Hook-and-loop fasteners are for example available from Velcro, USA.

Further examples of mechanical fasteners are button and holes or button loops, snap fasteners and the like. Combinations of adhesive and mechanical fasteners may also be provided.

The fasteners are intended to be fastened to a target area 18 on the first body panel 11 when the article is put on a wearer. The target area 18 is defined as the area of the first body panel 11 that is intended to receive the fasteners when the article is of an appropriate size for the wearer. The target area 18 is located at a selected distance from the transverse edge 11b of the first body panel 11 in a transverse central area thereof, which means the area centered over the transverse central axis 20 of the article. The target area 18 is preferably, but not necessarily, indicated by colour, character, graphic, text or combinations thereof. The dimension of the target area 18 may vary, but preferably it has an extension in transverse direction, x, that is at least 20% and more preferably at least 25%, of the width of the first body panel 11 in transverse direction. It is further preferred that the target area 18 has extension in transverse direction, x, that is not more than 70%, more preferably not more than 60%, of the width of the first body panel 11 in transverse direction.

The target area 18 comprises a reception material for the fasteners 17. A plastic film or a nonwoven material will function as a reception material for an adhesive tape, while for a hook fastener the reception material is a loop material as mentioned above.

The article further comprises size fit indicating means 19 arranged on the first body panel 11 at either lateral side of the target area 18, i.e. between the target area 18 and the respective longitudinal side edge 11a of the first body panel 11. The size fit indicating means 19 are intended to indicate when the article is too small for the wearer, who would need to change to an article of a larger size in order to have an adequate fit and security against leakage. The size fit indicating means 19 are indicated by colour, character, graphic, text or combinations thereof. In case the target area 18 is also indicated by colour, character, graphic, text etc. the indication of the size fit indicating means 19 should be different and distinguishable from the indication of the target area 18. Thus the target area 18 and the size fit indicating means 19 can be indicated by areas of different colour, different characters, different graphics, different text or combinations thereof. The size fit indicating means 19, i.e. the area on which size fit indicating means are arranged, may extend all the way from the target area 18 to the respective side edge 11a of the first body panel or may end a selected distance inside said side edge 11a. Preferably each size fit indicating means 19, i.e. the area on which size fit indicating means are arranged, has an extension in transverse direction, x, that is at least 10% and more preferably at least 15%, of the width of the first body panel 11 in transverse direction. It is further preferred that each size fit indicating means 18 has an extension in transverse direction, x, that is not more than 30%, more preferably not more than 25%, of the width of the first body panel 11 in transverse direction.

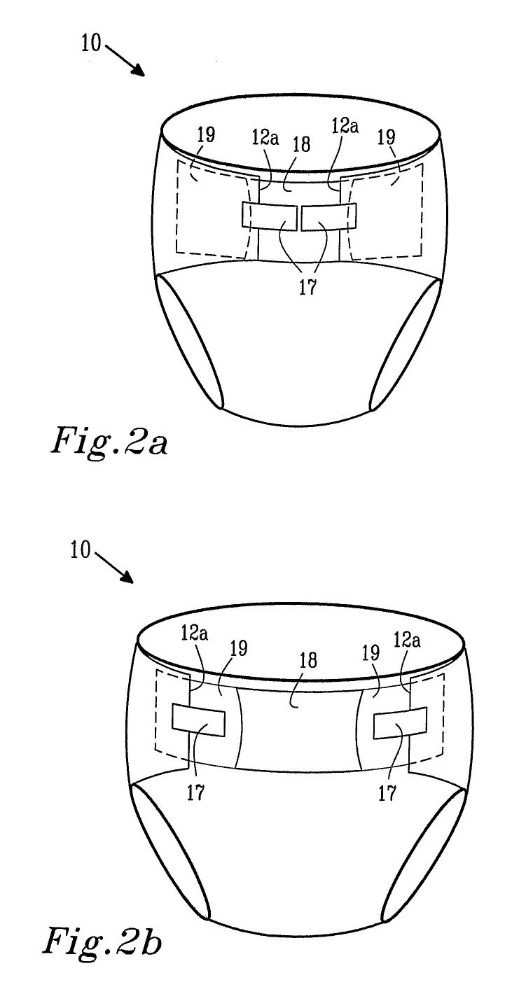

The size fit indication is intended to function in the following manner. When the article is fastened to a wearer, as illustrated in FIG. 2a, and the wearer is of a size that allows the fasteners 17 to be fastened to the target area 18 on the first body panel 11, the size fit indicating means 19 will become hidden behind the longitudinal side edges 12a of the second body panel 12. Thus FIG. 2a illustrates a configuration of the article when this is of a size that fits the wearer. However when the article is of a size that is too small for the wearer the fasteners 17 can not be attached to the target area 18, but will have to be fastened laterally outside thereof, thus exposing the size fit indicating means 19, which become visible between the target area 18 and the respective longitudinal side edge 12a of the second body panel 12. Such a configuration of the article is shown in FIG. 2b.

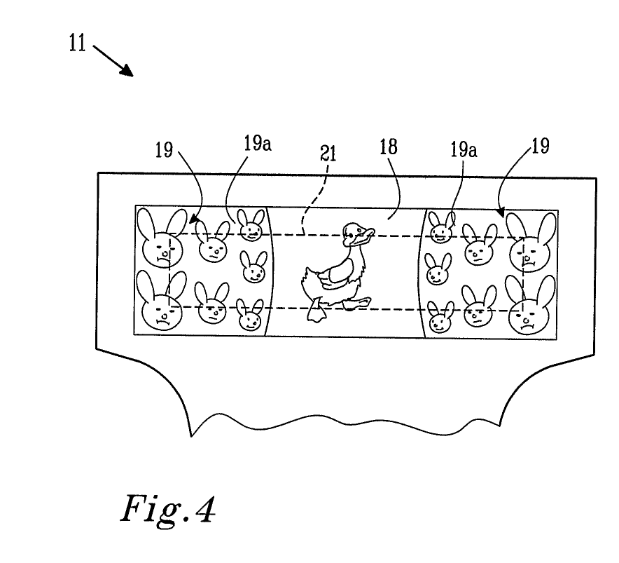

The target area 18 and the size fit indicating means 19 may comprise a pattern printed on the outer coversheet 16 of the article. In this case the reception material onto which the fasteners 17 are fastened may either be the outer coversheet itself or alternatively a separate strip 21 of reception material attached to the outer coversheet 16, see FIG. 4 This strip 21 should be sufficiently transparent to allow the pattern of the target area 18 and the size fit indicating means 19 to be visible there through. The strip 21 may be of the same size as, or smaller or larger than the combined target area 18 and size fit indicating means 19.

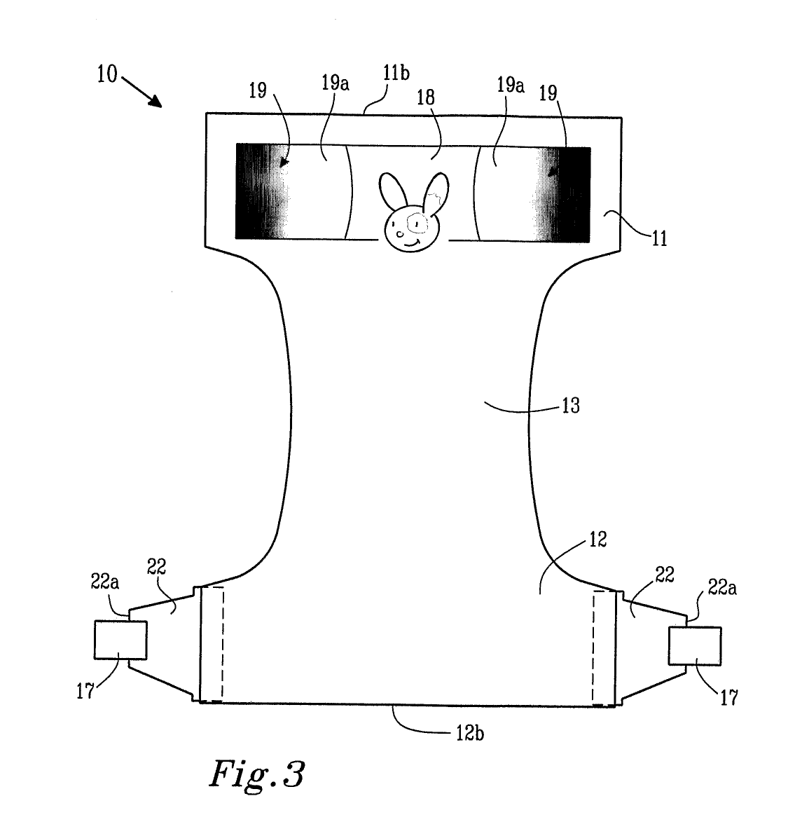

In FIG. 3 is shown an embodiment in which the size fit indicating means 19 comprises a transition zone 19a proximate to the target area 18, wherein the transition zone 19a forms a gradual indication of that the article is too small for the wearer. This can be accomplished by means of intensity of colour, size or pattern density of characters, graphic, text or combinations thereof. In FIG. 3 the transition zone 19a is formed by an increasing intensity of the colour of the size fit indicating means 19. In FIG. 4 the transition zone 19a is formed by an increasing size of characters in the form of rabbits. A further indication in the form of the expression of the rabbits from happy to sad is also illustrated in FIG. 4, wherein sad illustrates that the diaper is far too small.

In the embodiment shown in FIG. 3 the fasteners 17 are attached to the second body panel 12 via an elastic tab 22, which has an extension in longitudinal direction, y, that considerably exceeds the corresponding dimension of the fastener 17. In this case the longitudinal side edge of the second body panel 12 is defined as the longitudinal side edge 22a of the elastic tab 22, since when the article is fastened to the wearer the position of the longitudinal side edge 22a of the elastic tab 22 will be determining for whether the size fit indicating means 19 are exposed or not.

In one embodiment of the invention the article is of a selected size forming a part of an assortment of at least two different sizes of articles, such as small, medium, large, wherein each size has a selected indication in the form of colour, character, graphic, text or combinations thereof. The size fit indicating means 19 comprises at least one area having a colour, character, graphic, text or combinations thereof which is an indication of a size larger than the size of the article itself, i.e. the next size in the assortment. If for example the article is of the size “small” indicated by yellow colour, the size fit indicating means 19 can have at least one area thereof indicating the size “medium”, for example indicated by blue colour. This means that the caregiver in addition to being informed of that the article is too small is also informed of to which size the change should be made.

In FIG. 4 the target area 18 has a character in the form of a bird printed thereon, wherein the bird may be an indication of the size of the article. The size fit indicating means 19, on the other hand, has rabbits printed thereon, wherein the rabbit is indicative for the next size in the assortment.

Although only a few exemplary embodiments have been described in detail above, those skilled in the art will readily understand that many modification are possible. Any such modifications are intended to be included within the scope of this invention, which is defined in the claims below.

1. An absorbent article comprising a first body panel, a second body panel and a crotch portion extending between the first and second body panels, said article having a longitudinal direction and a transverse direction and said first and second body panels having longitudinal and transverse side edges, said first body panel comprises a target area adapted to receive fasteners located at the opposing longitudinal side edges of the second body panel, wherein when said fasteners are fastened to said target area the article will assume a pant-like shape, and wherein at least one size fit indicator is arranged on the first body panel, wherein said target area is located in a transverse central part of the first body panel and said size fit indicator is located between said target area and a longitudinal side edge of the first body panel and is adapted to indicate that the article is too small for the wearer.

2. An absorbent article as claimed in claim 1, wherein said article is of a selected size forming a part of an assortment of at least two different sizes of articles wherein each size has a selected indication in the form of colour, character, graphic, text or combinations thereof, said size fit indicator comprises at least one area having a colour, character, graphic, text or combinations thereof being an indication of a size larger than the size of the article itself.

3. An absorbent article as claimed in claim 1, wherein the target area and the size fit indicator are arranged so that when, in use, the article is fastened to a wearer by means of said fasteners, the respective longitudinal side edge of the second body panel, to which the fastener is attached, will be located laterally inside the size fit indicator to substantially hide the size fit indicator when the size of the article is adequate for the wearer, but when the size of the article is too small for the wearer, the size fit indicator will become visible between the target area and said longitudinal side edge of the second body panel.

4. An absorbent article as claimed in claim 1, characterized in that size fit indicator are arranged on both lateral sides of said transverse central part forming the target area.

5. An absorbent article as claimed in claim 1, wherein said size fit indicator comprises a transition zone proximate to said target area, wherein said transition zone forms a gradual indication of that the article is too small for the wearer by means of intensity of colour, size, pattern density or expression of characters, graphic, text or combinations thereof.

6. An absorbent article as claimed in claim 1, wherein the size fit indicator comprises a pattern printed on a garment facing surface of the article.

7. An absorbent article as claimed in claim 6, wherein a strip of reception material adapted to receive said fasteners is arranged on said first body panel, said strip extending in a lateral direction over said target area (18) and over at least a part of the size fit indicator (19), said strip of reception material being transparent so that said printed pattern is visible there through.

8. An absorbent article as claimed in claim 1, wherein the target area is located on a strip of reception material adapted to receive said fasteners.

9. An absorbent article as claimed in claim 8, wherein the size fit indicator comprises a pattern printed on said strip of reception material.

10. An absorbent article as claimed in claim 6, wherein the garment facing surface of the article acts as a reception material adapted to receive said fasteners.

11. An absorbent article as claimed in claim 1, wherein the target area comprises a pattern or colour distinctly different from that of the size fit indicator.

12. An absorbent article as claimed in claim 1, wherein said article is a baby diaper.
